(19) United States
(12) Patent Application Publication
(10) Pub. No.: US 2010/0114311 A1
(43) Pub. Date: May 6, 2010

(54) Multi-Lumen Breast Prothesis and Improved Valve Assembly Therefor

(76) Inventor: Hilton Becker, Boca Raton, FL (US)

Correspondence Address: LOWE HAUPTMAN HAM & BERNER, LLP, 1700 DIAGONAL ROAD, SUITE 300, ALEXANDRIA, VA, 22314, US

(21) Appl. No.: 12/496,365

(22) Filed: Jul. 1, 2009

Related U.S. Application Data
(60) Provisional application No. 61/111,440, filed on Nov. 5, 2008.

(51) Int. Cl. A61F 2/12

(52) U.S. Cl. 623/8

(57) ABSTRACT

A valve assembly for a mammary implant having a chamber defined by a flexible membrane includes a valve and a flexible filling tube which includes a relatively short semi-riged tubular structure that extends into the chamber and defines a passageway. The filling tube comprises a soft and flexible length of tubing and a solid portion that has an outside diameter that is slightly larger than the inner diameter of the semi-riged tube and is stretchable longitudinally to reduce its outer diameter to facilitate passage through the semi-riged tube. The solid portion includes a T-shaped flange at its inner end and is adapted to sealing the engaged semi-riged tube upon relaxation thereof to thereby form a plug seal. The plug seal includes a plurality of reverse barbs to prevent the plug seal from becoming dislodged and forced back into the chamber.

### CROSS-REFERENCE TO RELATED APPLICATIONS

This application claims the benefit of the filing date of U.S. Provisional Application Ser. No. 61/111,440, filed Nov. 5, 2008, entitled “Multi-Lumen Breast Prosthesis and Improved Valve Assembly Therefrom”.

### FIELD OF THE INVENTION

This invention relates to a multi-lumen breast prosthesis and improved valve and seal assembly for such prosthesis and more particularly to a multi-lumen breast prosthesis of the type having an outer lumen, a mass of gel disposed in the outer lumen and an inner lumen and a mass of saline disposed in the inner lumen. The invention also relates to an improved valve and seal assembly which includes means for preventing a plug valve from being dislodged and moving inwardly toward the interior of a lumen.

### BACKGROUND FOR THE INVENTION

Dual lumen breast implants for insertion into a human body are well known and have been in use for a number of years. For example, one of my earlier U.S. Pat. No. 6,183,514 defines a self-positioning breast implant. As described, a dual lumen gravity oriented breast prosthesis or implant for surgical implantation includes inner and outer lumens which are defined by inner and outer closed envelopes of a medical grade elastamer. The outer closed envelope or lumen defines a generally oval or tear shape with upper and lower portions. The outer envelope also contains a quantity of fluid material as for example a silicone gel having a first density. The prosthesis also includes a second or inner lumen defined by a second closed envelope of medical grade elastamer which is disposed in and fixed in the lower portion of the outer closed envelope. The inner lumen may be of various shapes such as generally spherical or preferably crescent shape. The second lumen is filled with a saline solution which has a density which is greater than the density of the silicone gel. The inner lumen with its greater density maintains or returns the prosthesis to its predetermined orientation on an individual in whom the prosthesis is implanted when the individual is in a standing or sitting position.

A more recent development in mammary prosthesis is disclosed in my more recent Patent on An Adjustable Gel Filled Mammary Prosthesis, U.S. Pat. No. 7,081,136. As disclosed therein, an adjustable gel filled mammary implant includes inner and outer envelopes with the outer envelopment containing about 95% of its volume filled with a silicone gel or the like. The inner envelope is attached to a bottom portion of the outer envelope and defines a volume of about 5% to 10% of the volume of the outer envelope and is filled with saline. The implant also includes a self-sealing valve, filling tube and reservoir for adjusting the size of the implant.

For a more natural appearing beast, it is desirable to have the two lumens move independently of each other. This is particularly important in implants having a predominant amount of gel in an outer lumen with a relatively small lumen that can be filled to 10 to 20% (preferably) of the volume of the outer lumen with a saline solution. The small saline inner lumen which is free floating will move naturally within the breast. Further, as the breast is naturally pear shaped, the filled saline chamber will give the implant an anatomical or pear shape to simulate a natural breast.

Due to prior difficulties in manufacturing, the smaller inner lumen and outer lumen are adhered together with a single valve allowing saline to be added to the inner lumen. Although functional, the result in shape is not optimal. One and probably the best approach to overcome this problem was to use a filling tube and seal construction in accordance with my earlier U.S. Pat. No. 5,507,808. As described therein an inflatable implant for use in human breast reconstruction includes a chamber which is defined by a flexible membrane. The implant also includes a valve and a flexible filling tube which passes through the valve and into the chamber.

The valve comprises a short semi-riged tube that surrounds an opening in the membrane and extends inwardly of the membrane and into the chamber. The semi-riged tube defines a passageway extending therethrough and also defines a relatively small reservoir which opens into the passageway. A filling tube comprises a soft and flexible length of tubing and a solid portion that has an outer diameter which is slightly larger than the inner diameter of the semi-riged tube and is stretchable longitudinally to reduce its outer diameter to facilitate passage through the semi-riged tube and the solid portion is adapted to sealingly engage the semi-riged tube upon relaxation thereof. There are two problems with this approach when making a multi-lumen implant wherein the inner lumen is free floating within the outer lumen. The problems are that the filling tube and sealed construction that includes a small reservoir for a better seal are relatively large and expensive to manufacture. Second, the plug portion of the valve occasionally dislodges and moves backwardly into the inner lumen allowing a leak in the inner lumen.

Therefore it is presently believed that there is a need for and potential commercial market for an improved multi-lumen implant with an improved filling tube and seal construction in accordance with the present invention. There should be a market for such implants because the use of a smaller valve allows for a smaller inner lumen as well as a significant reduction in cost. Further, it is presently believed that the new valve and seal will reduce if not eliminate problems of the plug valve becoming dislodged.

### BRIEF SUMMARY OF THE INVENTION

In essence, the present invention contemplates an improved filling tube and seal construction for a multi-lumen breast prosthesis of the type having an outer lumen or chamber and a mass of gel disposed within the outer lumen. The prosthesis also includes an inner lumen or chamber and a mass of saline disposed within the inner lumen. The outer lumen is defined by a flexible membrane with an inlet opening and a first semi-riged tube surrounded by the inlet opening and extending inwardly into the outer chamber. The first semi-riged tube defines a longitudinally extending passageway with a near end adjacent to, abutting and sealed to the inlet opening to close the inlet opening except for the longitudinally extending passageway. The far end of the first semi-riged tube extends into the outer lumen or chamber.

A filling tube comprising a soft and flexible length of tubing with a solid portion and a T-shaped flange at its distal end. The solid portion of the filling tube is slightly longer than the length of semi-riged tube while the remaining portion of the filling tube defines a second hollow passageway with an opening for connecting the interior of the filling tube with the interior of the chamber for inflation of the chamber. The filling tube has an outer dimension that is slightly larger than the inner dimension of the semi-riged tube and is stretchable longitudinally to reduce the outer diameter to facilitate passage through the passageway in the semi-riged tube. The solid portion of the filling tube sealingly engages the semi-riged tube upon relaxation thereof but can be stretched longitudinally and moved outwardly through the semi-riged tube by pulling on the proximal portion until the T-shaped flange engages the top of the semi-riged tube. A key element in the present invention resides in means such as a plurality of reverse barbs for preventing a plug valve from being dislodged or moved inwardly into the interior of the inner lumen. These barbs are typically disposed between the solid portions of the filling tube and the semi-riged tube. For example, a plurality of rearwardly directed needle like elements or the like disposed in the solid portion of the filling tube allow the filling tube to move into a constricted area of the semi-riged tube but once pulled into place prevent the remaining plug valve from rearward movement. In effect the solid plug is blocked from movement by the barbs in one direction and by the T-shaped flange in the other direction.

In a preferred embodiment of the invention the improved filling tube and seal construction are used for the inner chamber that is filled with saline while a second conventional flap valve is used to close the outer chamber that contains a more viscose silicone gel. This gel is less likely to leak in view of its higher viscosity. Nevertheless, it is contemplated that the improved sealing tube and seal construction may be used for both the interior and outer chambers.

The invention will now be described in connection with the accompanying drawings wherein like reference numerals have been used to define like parts.

### DESCRIPTION OF THE DRAWINGS

FIG. 1 is a perspective view illustrating a single lumen inflatable prosthesis embodying a filling tube and seal construction as shown in FIGS. 2-6 and shown with a filling tube and an attached injection dome in place for inflating the prosthesis;

FIG. 2 is an enlarged view of a portion of FIG. 1 showing the filling tube in position for inflating the prosthesis;

FIG. 3 is an enlarged view of a portion of FIG. 1 showing a closed filling tube;

FIG. 4 is an enlarged view of the filling tube and sealing construction according to one embodiment of the invention;

FIG. 5 is a perspective view of a plug seal in accordance with a preferred embodiment of the present invention;

FIG. 6 is a cross-section view of a plug seal in accordance with a further embodiment of the invention;

FIG. 7 is a schematic cross-sectional view of a breast with a dual lumen mammory prosthesis implanted therein with a dual valve in accordance with one embodiment of the invention;

FIG. 8 is a schematic illustration of the dual valve arrangement in an inner and outer lumen wherein the valve is shown as partially closed and a second plug is shown near closure;

FIG. 9 is a schematic illustration of a dual lumen implant with inner and outer plug valves in accordance with the invention;

FIG. 10 is a schematic illustration of a dual lumen implant with a plug valve, a leaf valve and a filling tube in accordance with the invention;

FIG. 11 is schematic illustration of a dual lumen implant as shown in FIG. 10 but with the filling tube removed;

FIG. 12 is a schematic illustration of a dual lumen implant in accordance with a further embodiment of the invention wherein a thin tube connects the inner lumen to the exterior of the outer lumen and wherein a single plug valves acts as a seal for the inner and outer lumen;

FIG. 13 is a schematic illustration of the dual lumen implant shown in FIG. 12 but with the filling tube removed; and

FIG. 14 is a schematic illustration of the further embodiment of the invention shown in FIGS. 12 and 13 within a human breast that illustrates the connection between the inner lumen and the outer lumen and that allows limited movement of the inner lumen while providing for a more natural shape.

### DESCRIPTION OF THE PREFERRED EMBODIMENTS OF THE INVENTION

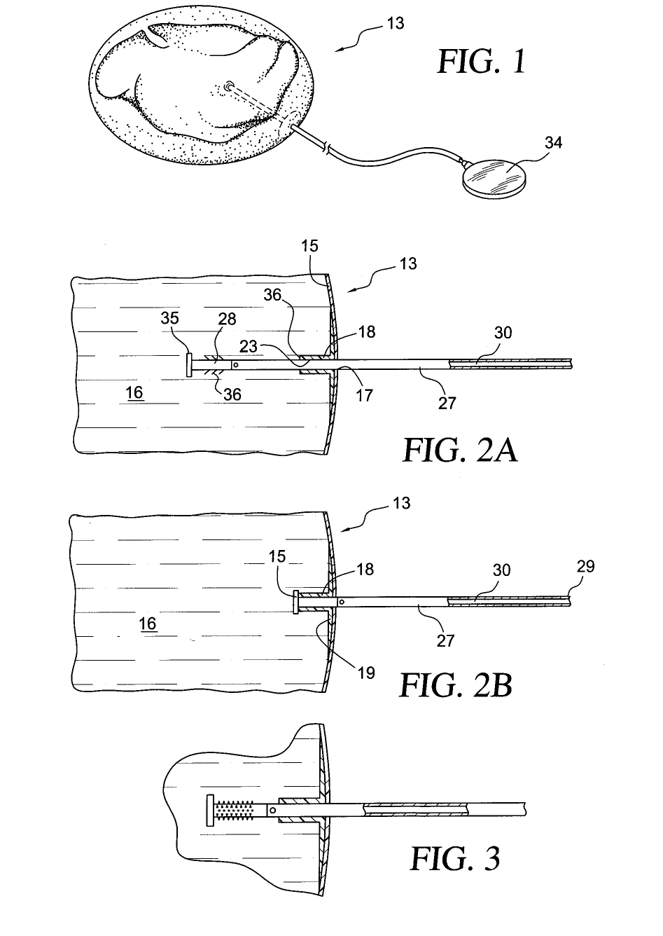

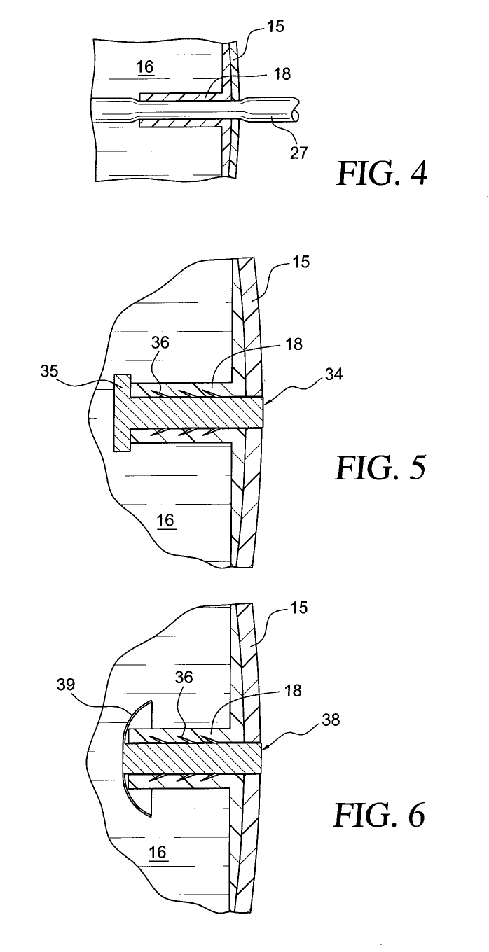

References will now be made in detail to the embodiments of the invention, examples of which are illustrated in the accompanying drawings. A preferred embodiment i.e. best mode of the filling tube and seal construction is shown in FIGS. 1-3. In accordance with the invention a filling tube and seal construction is embodied in an inflatable device such as an inflatable implant 13 which includes at least one flexible membrane or lumen 15. The flexible membrane or lumen 15 defines an inflation chamber 16 which is adapted for filling with a fluid to thereby inflate the implant 13. The lumen 15 also defines an inlet opening 17 to which an inflating fluid is delivered (See FIG. 2).

In accordance with the invention, the filling tube and seal construction includes a semi-rigid tube 18 which has a relatively thick wall with respect to the thickness of the membrane and which surrounds the opening 17. The semi-riged tube 18 extends inwardly of the lumen 15, and has a passage 23 therethrough which opens inside of the lumen 15.

The lumen 15 is constructed of a suitable material or membrane such as a medical grade silicone elastomer or similar material which does not react with human tissue, as will be understood by those skilled in the art. And, the short semi-rigid tube 18 may be formed of the same or of a different material but is preferably formed as an integral part of the membrane and derives its semi-rigid characteristic from the thickness of its wall section. It may also be desirable to form a thicker portion 19 of the lumen 15 as illustrated to further support the tube 18. This thickened portion 19 surrounds the opening 17 and reduces the likelihood of any breaking or tearing of the membrane. The semi-rigid tube 18 also defines an inner passageway or passage 23

A filling tube 27 constructed of silicone tubing or the like is adapted to pass through the passage 23 which is defined by the relatively short, semi-rigid tube 18. The filling tube 27 also includes a solid distal portion 28, which is preferably slightly longer than the length of the semi-rigid tube 18, and a proximal portion 29 which defines a hollow passageway 30 and an opening 32. The opening 32 connects the passageway 30 with the interior of the chamber 16 and operatively connects the interior of the chamber 16 to an injection dome or reservoir 34.

The filling tube 27 is relatively soft so as not to puncture or damage the tube 18 or the lumen 15. This filling tube 27 has an outer diameter which is slightly larger than the inside diameter of the passageway 23 and is stretchable in the longitudinal direction to reduce its outside diameter. Reduction of the outside diameter facilitates the passage of the filling tube 27 through the semirigid tube 18. And the solid portion 28 of the filling tube is adapted to sealingly engage the passageway 23 in the semi-rigid tube 18 upon relaxation from a stretched condition.

In practice, the filling tube may be forced through the passageway 23 by means of a rigid member (not shown) or could be molded into place by having the lumen and semi-rigid tube molded around the filling tube. In the latter case, the filling tube would include an area of reduced diameter so that the inside diameter of the tube 18 would be smaller than the outside diameter of at least the solid portion 28 of the filling tube 27.

The proximal end 27′ of the tube 27 is adapted for connection to a fluid source such as the reservoir 34. Fluid such as a saline solution is then forced from the reservoir 34 by means of a hypodermic needle inserted therein (not shown) through the passageway 30 and opening 17 and into the chamber 16. When the chamber 16 is sufficiently filled, the filling tube is stretched longitudinally by pulling and passes through the passageway 23 until the solid distal portion 28 is encompassed by the semi-rigid tube 18. When the pressure or longitudinal extension is released, the solid distal portion returns to its original outside diameter sealingly engages the semi-rigid tube 18.

In a preferred embodiment of the invention, the solid distal portion 28 includes a generally radially extending flange 35 having a generally T-shaped cross section for engaging a seat 36 on the semi-rigid tube 18. This radially extending flange acts as a stop means and prevents the filling tube 27 from being pulled through the semi-rigid tube 18 and contributes to the reliability of the seal.

Various factors must be considered in constructing the filling tube and seal or valve, such as the thickness of the semi-rigid tube, the stiffness of the materials, the lengths of the semi-rigid tube and the filling tube, their diameters including the nature and/or viscosity of the fluids contained within the implant, of the sealant in the reservoir, the pressure of the fluid, etc. Those skilled in the art will appreciate that the seal must not leak and must not cause discomfort to the patient.

It will also be appreciated that inflatable prosthesis of this type often have the filling tube inserted in place at the time the prosthesis is manufactured and prior to packaging. Thus, the filling tube remains in place during the entire shelf life of the prosthesis which can be for a considerable length of time. A number of previous valves acquired a “set” during this time so that when the prosthesis was implanted, expanded, and the filling tube removed, leakage occurred at the valve. This problem was remedied by incorporating a kink or curled valve into the system. However, the complexity of manufacture adds to the cost of the device. Furthermore, the curled valve can often be felt by the patient. It is presently thought that because of the small size of the valve and seal according to the present invention will overcome this tendency and as such constitutes an improvement on the valves disclosed in my earlier patent.

FIG. 5 is an enlarged view of a plug valve 34 in accordance with a preferred embodiment of the invention. As shown, the plug valve 34 is formed from the distal end of the solid portion 28 of the filling tube 27 and thus has a generally circular cross-section with the outwardly extending valve 35 at the top of the plug valve 34 to provide a T-shaped cross-section. The plug portion of the plug valve 34 also includes a plurality of rearwardly directed barbs 36. In other words, the barbs 36 in a preferred embodiment of the invention are pointed rearwardly i.e. toward the interior of a chamber and form an acute angle with a longitudinal axis of a semi-riged tube. With the longitudinal axis of the plug valve with this design, the barbs are pressed down into the soft filling tube as the plug is pulled into the semi-riged tube, but spring upwardly and penetrate into the semi-riged tube if the plug is moved backwards. An alternative plug valve 38 includes a flap-like closure 39 on its end that opens up to prevent the plug valve from being forced out of the semi-riged tube.

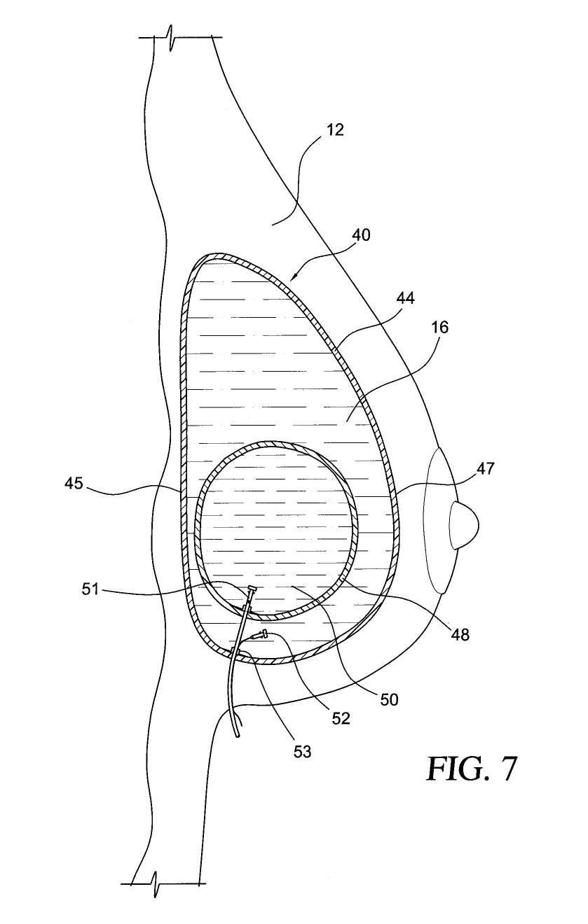

A dual lumen mammary prosthesis in accordance with a preferred embodiment of the invention is shown in FIG. 7. As shown, in FIG. 7, a mammary prosthesis or breast implant 40 is implanted in the breast tissue 12 of a patient. The implant 40 includes an outer envelope 44 of a medical elastomer such as silicone and a silicone gel is contained within the outer envelope 44. The silicone gel 16 may be replaced by other suitable material such as polyvinyl pyrralidone, hyaluronic acid, polyacrlimids and polysacckarides.

The walls of the lumen i.e. the outer envelope 44 and inner envelope 48 may be made of various soft flexible biocompatible materials such as silicone elastomer. Preferred materials include silicone elastomers such as polydimethylsiloxane or polymethylvinyl siloxane or copolymers thereof with other substances. Other polymers may be substituted as will be apparent to persons of ordinary skill in the art.

As illustrated in FIG. 7, the outer envelope 44 has an oval or generally tear-shape with a relatively flat rear portion 45 and rounded dome or forward surface 47. The outer envelope 44 defines an outer lumen which may be of a generally tear-drop shape or other non-symmetrical shape in order to conform to the contours of a human breast. It should be recognized that in certain cases a round shape may be needed.

The implant 40 also includes an inner lumen which is defined by an inner envelope 48. This inner envelope 48 is preferably of the same medical grade elastomer as the outer envelope 44. The inner envelope 48 may be of a substantially spherical shape and upon filling is preferably free floating within the outer envelope. The inner lumen or inner envelope 48 also contains a fluid 50 such as silicone solution which has a density greater than the density of the gel in the outer lumen. As illustrated, the implant 40 includes a pair of valves 51 and 52, one valve 51 for the inner envelope 48 and the other valve 52 for the outer envelope 44. As shown, the valves 51 and 52 are each plug valves with the valve 51 closing the inner envelope and the plug valve 52 attached to a nylon filament that extends from and positions the valve 52 to be drawn into a semi-riged tube 53.

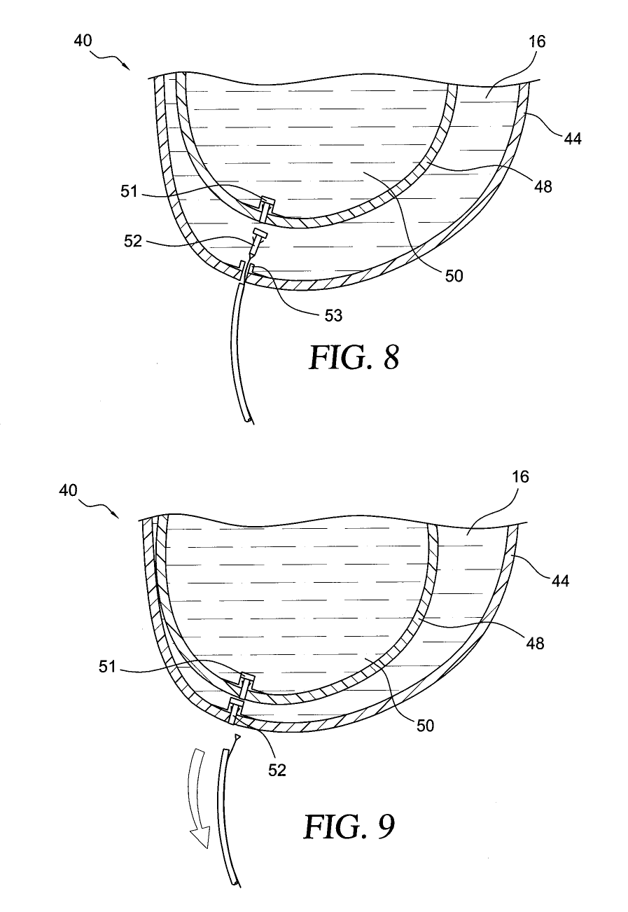

FIGS. 8 and 9 show the positioning of the two valves 51 and 52 as positioned for adjusting the amount of saline 50 in the inner chamber 48 and before the inner envelope 48 is allowed to float or move freely within the outer chamber and with the fill tube removed through the valve 52.

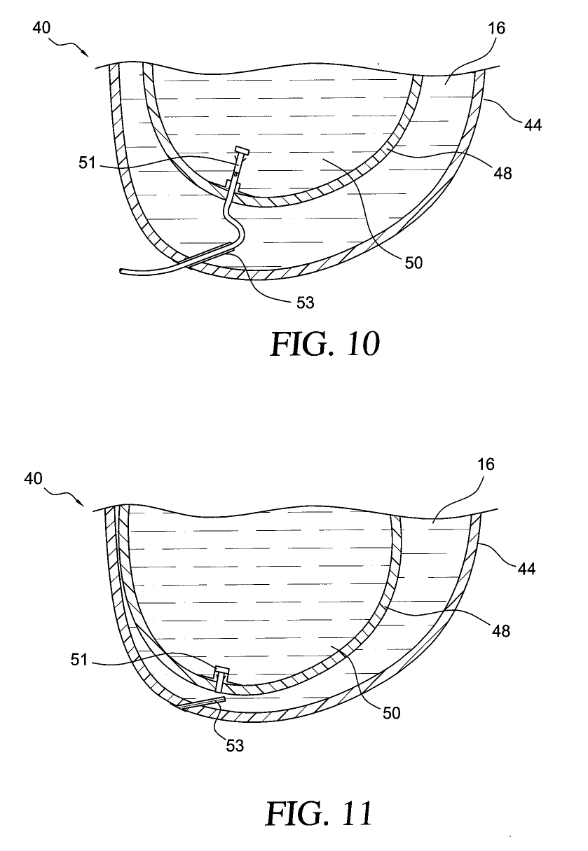

A further embodiment of the invention is illustrated in FIGS. 10 and 11 wherein a dual lumen implant includes an inner plug valve 51 in accordance with the present invention for sealing the inner lumen and a leaf valve 53 in the outer lumen. FIG. 10 illustrates the valves and filling tube in place while FIG. 11 illustrates the valves 51 and 53 in a closed position with the filling tube removed.

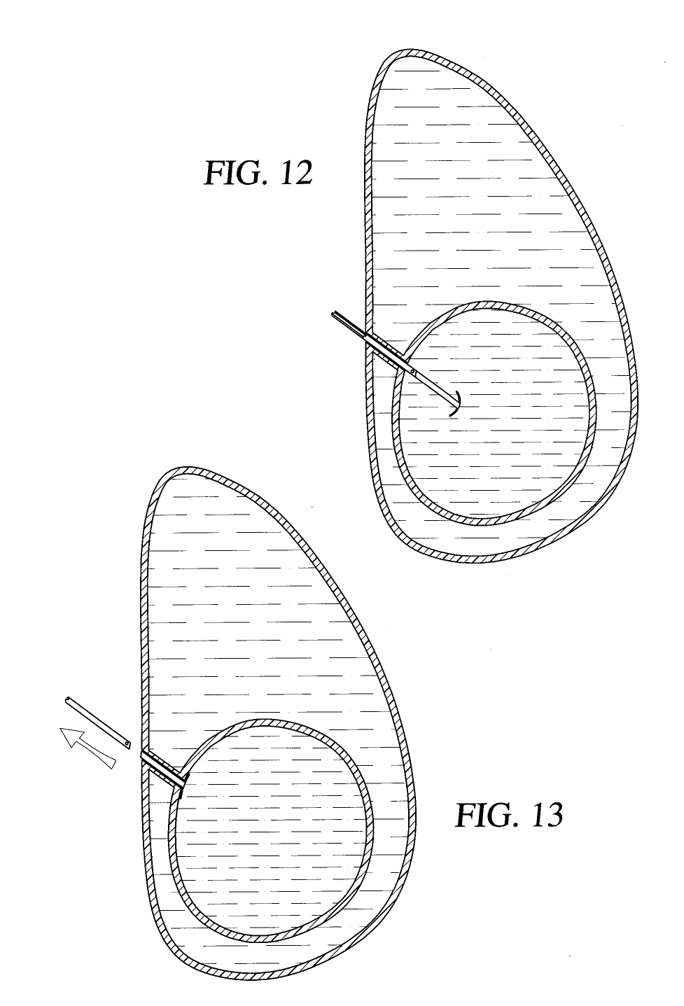

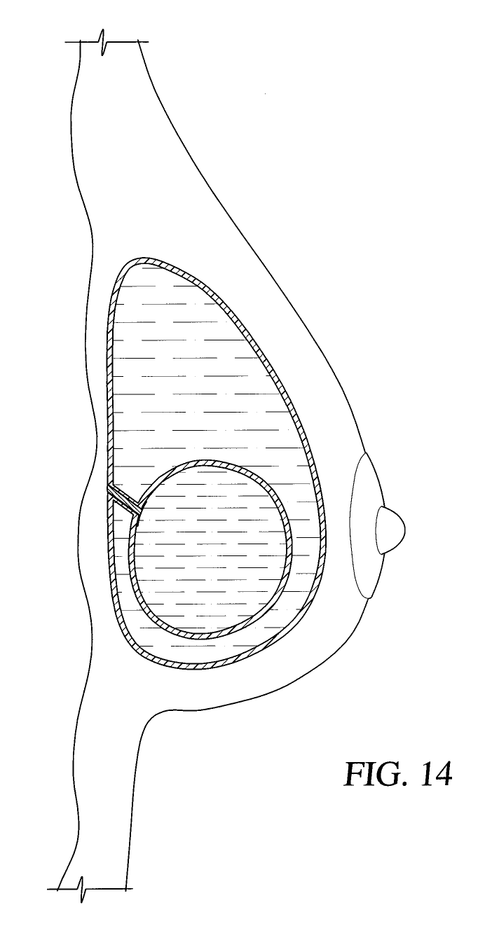

FIGS. 12 and 13 illustrate a still further embodiment of the invention wherein an outer envelope and an inner envelope are sealed by a single inner valve 51 and cooperates with a connecting member 68 to thereby maintain the fluid 50 within the inner lumen and at the same time close the chamber 16.

While the invention has been disclosed in connection with its preferred embodiments it should be recognized that changes and modifications may be made therein without departing from the scope of the claims.

What is claimed is:

1. A filling tube and seal construction for a multi-lumen breast prosthesis of the type having an outer lumen, a mass of gel disposed in said outer lumen, an inner lumen and a mass of saline disposed in said inner lumen, said prosthesis comprising:
an outer chamber defined by a flexible membrane and having an inlet opening in said chamber and a semi-riged tube surrounded by said inlet opening and extending inwardly into said chamber, said semi-riged tube defining a longitudinally extending passageway with a near end adjacent to and abutting said inlet opening and a far end within said chamber;
a filling tube comprising a soft and flexible length of tubing forming a solid portion at a distal end thereof, said solid portion being longer than said semi-riged tube, and the remaining portion of said filling tube defining a second hollow passageway and an opening for connecting the interior of said filling tube with the interior of said chamber for inflation of said chamber;
said filling tube having an outer diameter larger than the inner diameter of said semi-riged tube and being stretchable longitudinally to reduce its outer diameter to facilitate passage through said semi-riged tube, said solid portion of said filling tube sealingly engaging said semi-riged tube upon relaxation thereof whereby said filling tube can be stretched longitudinally and moved outwardly through said semi-riged tube by pulling on a proximal portion of said filling tube after inflation of said chamber to position said solid portion in said semi-riged tube; and
means disposed between said solid portion of said filling tube and said semi-riged tube to prevent retraction of said solid portion into the interior of said chamber.

2. A filling tube and seal construction for a multi-lumen breast prosthesis according to claim 1 in which said means disposed between said solid portion of said filling tube and said semi-riged tube include a series of barbs.

3. A filling tube and seal construction for a multi-lumen breast prosthesis according to claim 2 in which said barbs are stainless steel.

4. A filling tube and seal construction for a multi-lumen breast prosthesis according to claim 3 in which said barb forms an acute angle with respect to the longitudinal axis of said solid portion of said filling tube and points in a direction of said inner chambers.

5. A filling tube and seal construction for a multi-lumen breast prosthesis according to claim 1 in which said means disposed between said solid portion of said filling tube and said semi-riged tube include a plurality of particles disposed on or in a surface of one of said filling tubes and said semi-riged tube to thereby position said particles between said filling tube and said semi-riged tube.

6. A filling tube and seal construction for a multi-lumen breast prosthesis according to claim 5 in which said particles are abrasive particles.

7. A filling tube and seal construction for a multi-lumen breast prosthesis according to claim 1 in which the inner surface of one of said passageways and said semi-riged tube and an outer surface of said solid portion of said filling tube is textured.

8. A filling tube and seal construction for a multi-lumen breast prosthesis according to claim 1 in which said filling tube and said semi-riged tube are made of a silicone elastomer.

9. A filling tube and seal construction for a multi-lumen breast prosthesis according to claim 1 in which said filling tube includes means at the distal end thereof, engageable with said semi-riged tube to limit withdrawal of said filling tube and means for limiting withdrawal of said filling tube comprises a radially extending flange having a generally T-shaped cross section.

10. A filling tube and seal construction for a multi-lumen breast prosthesis according to claim 1 in which said filling tube is made of silicone elastomer.

11. A filling tube and seal construction for a multi-lumen breast prosthesis according to claim 1 in which said membrane includes a thicker portion adjacent said semi-riged tube to thereby reduce the likelihood of tearing the membrane in that area.

12. A multi-lumen breast prosthesis of the type having an outer lumen, a mass of gel disposed in said outer lumen, an inner lumen and a mass of saline disposed in said inner lumen, said prosthesis comprising:
an outer chamber defined by a flexible membrane and having an inlet opening in said chamber and a first inwardly projecting semi-riged tube surrounded by said inlet opening and extending inwardly into said outer chamber, said first inwardly projecting semi-riged tube defining a longitudinally extending passageway with a near end adjacent to and abutting said first inlet opening and a far end within said outer chamber;
an inner chamber defined by a separate flexible membrane and having an inlet opening in said chamber and a second or separate inwardly projecting semi-riged tube surrounded by said inlet opening of said inner chamber and extending inwardly into said inner chamber, said separate inwardly projecting semi-riged tube defining a longitudinally extending passageway with a near end adjacent to and abutting said inlet opening in said inner chamber and a far end within said inner chamber;
a filling tube comprising a soft and flexible length of tubing forming a solid portion at a distal end thereof, said solid portion of said flexible tubular member having a larger diameter than said separate inwardly projecting semi-riged tube and including an outwardly extending flange for engaging an inner end of said separate semi-riged tube to thereby form a first plug valve for closing said passage through said separate semi-riged tube, and the remaining portion of said filling tube defining a third hollow passageway and an opening for connecting the interior of said filling tube with the interior of said inner chamber for inflating or partially deflating said inner chamber, and said filling tube having an outer diameter larger than the inner diameter of said separate semi-riged tube and being stretchable longitudinally to reduce its outer diameter to facilitate passage through said separate semi-riged tube and said solid portion of said filling tube sealingly engaging said separate semi-riged tube upon relaxation thereof whereby said filling tube can be stretchable longitudinally and moved outwardly through said separate semi-riged tube by pulling on a proximal portion of said filling tube after inflation of said chamber and means for preventing said solid portion of said filling tube from becoming dislodged or forced into said inner chamber;
a second valve for closing said passageway in said first semi-riged tube after removal of said filling tube; and
means disposed between said solid portion of said filling tube and said separate semi-riged tube to prevent dislodging or retraction of said solid portion into the interior of said inner chamber.

13. A multi-lumen breast prosthesis according to claim 12 in which said means includes a series of projections between said solid portion and said filling tube includes a plurality of barbs to prevent said solid portion of said filling tube from being forced into said inner chamber.

14. A multi-lumen breast prosthesis according to claim 12 in which said barbs are stainless steel and form an acute angle with said solid portion and point in the direction of said inner chamber.

15. A multi-lumen breast prosthesis according to claim 12 in which said barbs are abrasive particles.

16. A multi-lumen breast prosthesis according to claim 12 in which said barbs comprise a textured surface.

17. A multi-lumen implant comprising an outer chamber and a mass of gel disposed in said outer chamber, an inner chamber and a mass of saline disposed in said inner chamber and a connector filling tube extending from said exterior wall of said outer chamber and into said inner chamber for filling or removing saline from said inner chamber and said connecting tube having a length of about ⅓ to about ⅔ the diameter of said inner chamber for limiting movement of said inner chamber within said outer chamber.
